(19) United States
(12) Patent Application Publication
(10) Pub. No.: US 2024/0361327 A1
(43) Pub. Date: Oct. 31, 2024

(54) DETECTION AND ANALYSIS OF CIRCULATING TUMOR CELLS

(71) Applicant: QCDx LLC, Farmington, CT (US)

(72) Inventor: Triantafyllos P. TAFAS, Rocky Hill, CT (US)

(21) Appl. No.: 18/595,623

(22) Filed: Mar. 5, 2024

Related U.S. Application Data
(63) Continuation of application No. PCT/US2022/012137, filed on Jan. 12, 2022.
(60) Provisional application No. 63/136,259, filed on Jan. 12, 2021.
(60) Provisional application No. 63/285,951, filed on Dec. 3, 2021.

(51) Int. Cl. G01N 33/574; G01N 15/10; G01N 15/14

(52) U.S. Cl. CPC G01N 33/57492; G01N 15/1468; G01N 2015/1006

(57) ABSTRACT

The present invention provides systems and compositions for detecting circulating tumor cells (CTCs), and methods thereof. The systems of the present invention can image (e.g., via planar imaging) one or more cells (e.g., embedded in a solid or semi-solid medium, such as a gel) and analyze the cells, to identify one or more CTCs from the one or more cells.

### CROSS-REFERENCE

This application is a continuation of International Patent Application No. PCT/US2022/012137, filed Jan. 12, 2022, which claims the benefit of U.S. Provisional Patent Application No. 63/136,259 filed on Jan. 12, 2021 and U.S. Provisional Patent Application No. 63/285,951 filed on Dec. 3, 2021, each of which is entirely incorporated herein by reference.

### BACKGROUND

Early stage and even small tumors can release cancer cells in blood that carry a signature in the form of circulating tumor cells (CTCs) and can be responsible for the creation of metastases. In some cases, cancer management can require frequent monitoring over time. Monitoring can be challenging, for example, when a remote tumor site precludes multiple repeat biopsies.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some embodiments, the invention provides a method for analyzing a biological sample obtained from a subject, the method comprising: (a) contacting a sample of cells of the biological sample with a plurality of detection moieties, wherein the plurality of detection moieties comprises (i) a first detection moiety exhibiting specific binding to a first target ligand, wherein the binding to the first target ligand results in staining, and (ii) a second detection moiety exhibiting specific binding to a second target ligand, wherein the binding to the second target ligand results in staining; (b) subsequent to (a), imaging the biological sample via selective plane image microscopy, to obtain a plurality of planar images of the sample of cells, wherein the plurality of planar images comprises: (i) a first image indicative of presence or absence of the first target ligand in the sample of cells based on staining or lack of staining by the first detection moiety, and (ii) a second image indicative of presence or absence of the second target ligand in the sample of cells based on staining or lack of staining by the second detection moiety; and (c) analyzing the plurality of planar images, to identify a diseased cell from the sample of cells.

In some embodiments, the invention provides a system for analyzing a biological sample obtained from a subject, the system comprising: a container for holding a sample of cells of the biological sample; an imaging unit configured to image the sample of cells disposed in the container via selective plane image microscopy; and a processor operatively coupled to the imaging unit, wherein the processor is configured to: (a) direct the imaging unit to image the sample of cells disposed in the container, to obtain a plurality of planar images of the sample of cells, wherein the plurality of planar images comprises: (i) a first image indicative of presence or absence of a first target ligand in the sample of cells based on staining or lack of staining of the first target ligand by a first detection moiety, and (ii) a second image indicative of presence or absence of a second target ligand in the sample of cells based on staining or lack of staining of the second target ligand by a second detection moiety; and (b) analyze the plurality of planar images, to identify a diseased cell from the sample of cells.

### BRIEF DESCRIPTION OF THE DRAWINGS

FIGS. 1A and 1B show assessment of mesenchymal cell and epithelial cell phenotype comparison in CTCs. FIG. 1A: Each dot represents one CTC from timepoints S1 (higher dots) and S2 (lower dots). Fluorescent intensity is the top grey level value recorded from the digital 3D image planes containing each CTC. FIG. 1B: A comparison of CK to Vim expressions between timepoints S1 and S2, unpaired Mann-Whitney nonparametric T-test (*=P<0.001). Range of grey level values is 0-255.

FIGS. 2A-2C show assessment of expression of Tumor-associated calcium signal transducer 2 (Trop2), Vimentin (Vim), and/or Cytokeratin (CK) in CTCs. FIG. 2A: Spearman correlation between expression levels for Trop2, Vim, and CK in timepoints S1 and S2 (*=P<0.001). FIG. 2B: Difference in Trop2 expression in CTCs between timepoints S1 and S2, statistically significant with unpaired Mann-Whitney nonparametric T-test for statistical analysis (*=P<0.001). FIG. 2C: Total CTC and Trop2+, Vim+& CK+ CTCs in S1 and S2. Percentages indicate relative CTC frequencies within each timepoint.

FIG. 3 shows images of CTCs with varying expression of Trop2, Vimentin, and Cytokeratin (scale: 10 micrometer).

FIG. 4 shows a drawing for the sample holder and of the sample handler of the present invention.

FIG. 5 schematically illustrates Fluorescence Light Sheet Microscopy Principle for imaging, e.g., three-dimensional (3D) optimal tomography.

FIG. 6 schematically illustrates an example process of analyzing a blood sample to detect CTCs.

FIGS. 7A and 7B show example immunofluorescent staining images of CTCs in the two aliquots: one used for diagnostic antibody panel (FIG. 7A), and the second used for the treatment antibody panel (FIG. 7B) (scale: 10 micrometer).

FIG. 8 shows characterization of diagnostic markers of circulating tumor cells in patients studied.

FIG. 9A shows total circulating tumor cell counts that change over time for individual patients, as ascertained by the systems and methods of the present disclosure.

FIG. 9B shows phenotypic changes in diagnostic and treatment panels for individual patients, as ascertained by the systems and methods of the present disclosure.

FIGS. 10A and 10B show detection of total CTCs across multiple time points, based on use of diagnostic markers (FIG. 10A) or treatment markers (FIG. 10B).

### DETAILED DESCRIPTION

The present invention provides systems and compositions for detecting circulating tumor cells (CTCs), and methods thereof. The systems of the present invention can image (e.g., via planar imaging) one or more cells (e.g., embedded in a solid or semi-solid medium, such as a gel) and analyze the cells, to identify one or more CTCs from the one or more cells. The compositions of the present invention can comprise one or more antibodies (e.g., a plurality of antibodies) to detect one or more cell types (e.g., a plurality of cell types, such as epithelical-like cell type and mesenchymal-like cell type), to identify one or more CTCs from a biological sample, such as a blood sample derived from a subject. The methods of the present invention can utilize any of the systems and compositions disclosed herein to identify one or more CTCs from a biological sample.

Identification CTCs, e.g., via a liquid biopsy, can be used to predict the characteristics of a tumor and for prognostication of cancer. The significance and presence of CTCs can be characterized and identified. For example, in breast cancer, CTCs can have an independent prognostic value in metastatic breast cancer (MBC) and early breast cancer. For example, if ≥5 CTCs are found per 7.5 milliliter (mL) of blood from a subject with breast cancer, this can be associated with poor prognosis for the cancer. CTC-count can improve the prognostication of MBC when added to full clinicopathological predictive models, which cannot be done with serum tumor markers. Single cell molecular characterization of isolated CTCs can provide detailed mapping of cancer cell clones from the initial and/or metastatic tumor sites. Longitudinally, molecular information from cancer cell clones resistant to treatment can be a more responsive method for treatment optimization, rather. than depending on the detection of new mutations in fragments of circulating tumor DNA.

In early breast cancer (EBC), CTC detection can be associated with a poor clinical outcome. Cytokeratin 19 (CK19)-mRNA can be used as a marker for CTC detection. Following detection, administration of Trastuzumab can reduce or eliminate chemotherapy-resistant CK19mRNA+ cells and improve patient outcome.

A CTC count (e.g., a CTC count of ≥5 per 7.5 mL of blood) at any time during the course of the disease can be associated with a poor prognosis and can be predictive of shorter Progression Free Survival (PFS) and Overall Survival (OS) in patients with metastatic breast cancer. TABLE 1 below lists median PFS and OS based on CTC counts.

TABLE 1

| Number of CTC | PFS (months) | OS (months) |
|---|---|---|
| At all-time <5 | 7.2 | 22.6 |
| Baseline <5; at final draw ≥5 | 5.9 | 10.6 |
| Baseline ≥5; at final draw <5 | 6.1 | 19.8 |
| At all-time points ≥5 | 1.8 | 4.1 |

A blood sample taken from a patient prior to a new line of therapy can be used for the baseline prediction while another sample taken at the first follow up visit can be used to predict whether the therapy is efficacious.

Human epidermal growth factor receptor 2 (HER2) evaluation at the DNA, mRNA, and protein level has been performed on CTCs. Although HER2+ CTC can be more commonly detected in women with HER2+ disease, in some women with HER2-breast cancer, HER2+CTCs are observed. In ER-positive MBC, CTC enumeration, phenotyping, and genotyping can identify patients who would benefit from Fulvestrant (selective estrogen receptor down-regulator) escalation versus switching to alternative therapies. CTCs are found in patients with inflammatory breast cancer (IBC), a highly aggressive form of breast cancer. CTCs can be found in IBC patients with abnormalities in adaptive immunity. Utilization of CTCs in patients with abnormalities in adaptive immunity could be a surrogate marker of a more aggressive disease with general immune system dysfunction.

Described herein is a liquid biopsy test to detect one or more CTCs by analyzing a plurality of peripheral blood mononuclear cells (PBMCs) (e.g., lymphocytes such as T cells, B cells, NK cells; monocytes) derived from a subject. The liquid biopsy test can be performed subsequent to enrichment of the plurality of PBMCs. In some embodiments, the liquid biopsy test does not require any enrichment of the plurality of PBMCs. In some embodiments, described herein is a liquid biopsy test to detect CTCs by analyzing PBMCs without prior enrichment. This personalized approach to analysis of CTCs can use various imaging methods, such as Selective Plane Illumination Microscopy (SPIM), to detect CTCs. In some embodiments, a method disclosed herein can use automated analysis to screen the entire PBMC population and identify CTCs based on staining (e.g., immunofluorescent staining) with positive or negative markers for one or more target cells, e.g., epithelial or mesenchymal phenotypes, single cells, clusters and/or apoptotic (dying) cells.

In some embodiments, CTC detection, including live characterization and characterization without enrichment, and using biomarkers for multiple phenotypes, can open the way for a standardized CTC definition and benefit precision cancer diagnosis.

The system as disclosed herein can permit ex vivo observation of cells (e.g., cells that have been stained with vital stains for CTC-specific biomarkers and maintained alive for periods of time) supported by a three-dimensional (3D) culture subsystem. In some embodiments, the system, can comprise a biological holder and a handler. A specially designed cell chamber can be fitted for input and output of culture media, gas regulation and control of environmental variables (temperature, pH etc). This can allow ex vivo observation of cells while perfused with culture media which may contain various substances. The chamber can be fitted with a micromanipulator (handler) used to isolate target cells under direct observation. Both the chamber and the micromanipulator can be operated automatically by a system computer and software system.

The ex vivo liquid biopsy can offer longitudinal observation of target cells, e.g. CTCs and/or white blood cells (WBCs) and assessment of desired and undesired toxicity of therapeutic drug cocktails before used for patient treatment. This can drive precision medicine for improved outcomes and reduced adverse effects to the patient. Cell isolation can enable CTC genomic and transcriptomic analysis that may reveal improved therapeutic options, tuned to the patient's current disease status.

The sample holder and handler of the present invention, combined with deep quantitation of every cell the specimen, can be a precision medicine tool. Deep CTC characterization and single-cell, genomic/transcriptomic analysis can enable the oncologist to select a treatment that is synchronized with the current disease stage. Ex vivo assessment of how a selected drug or drug combination affects CTCs and/or WBCs in the patient's blood can be assessed in view of patient outcomes.

In some embodiments, a central computer system operates a software package that (a) acquires and processes images of the biological specimen's features for identification and quantitation, (b) actuates the motorized components, pumps, sensors of the system, (c) operates a robotic arm that loads and unloads samples, and (d) handles digital information managed in local or wide area networks. The central computer system may utilize local or distributed processing protocols.

The system also includes or is coupled to a tunable laser source or multiple single wavelength laser sources, complete with light management optical path(s). An optical system modulating the light (e.g., light sheet, such as laser light sheet) can combine bilateral illumination to produce the sheet illumination for Selective Plane Illumination Microscopy (SPIM).

In some embodiments, imaging is performed by illuminating the specimen with narrow spectrum excitation light provided by monochromatic and/or tunable laser sources. Images of the resulting emission are acquired by high sensitivity monochrome cameras on a field by field basis. These images are combined in 3D stacks, which are then analyzed for quantitative measurement of biomarker levels in the individual cells. Alternatively, the images can be analyzed individually (e.g., without combining multiple images into a single image).

In operation, a biological specimen that can include live cells is stained with a variety of markers against proteins, nucleic acids or other cellular components and encased in an appropriately shaped cylindrical sheath to be fitted on a biological sample holder. The preparation is made by mixing the cell suspension with a solid or semi-solid medium (e.g., gels, such as agarose or other hydrogels that are compatible with preserving the subcellular structure of the embedded cells), at a temperature where the solution is still liquid. In addition to the cells, fluorescent beads that serve the role of fiducial reference for the identified cells are added to the solution. The liquid cell/bead/gel suspension is aspirated in tubing that is chosen to be transparent to the fluorescence light regime utilized. After being allowed to solidify, the specimen can be visualized in the light path. The biological specimen is mounted on a specimen holder loaded onto the microscope stage.

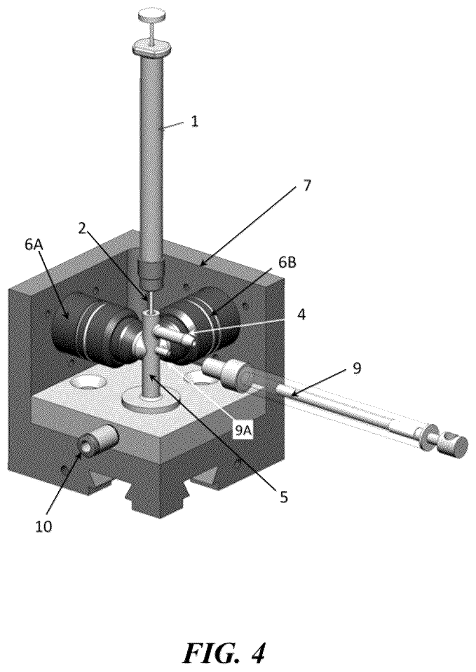

FIG. 4 shows a drawing for the sample holder and of the sample handler of the present invention. Shown is a means 1 for advancing and manipulating the sample 3 (not visible in this FIG. 4) contained within a sample holder such as a capillary tube 2 with a plurality of holes 2A (the capillary tube is not visible in this FIG. 4). The means 1 can be any of a variety of mechanical devices, including, for example a glass syringe. Shown is the cylindrical sample chamber 5, with a fluid output or outlet port 4, a lens holder 7, holding an illumination lens 6A and a detection lens 6B (which are oriented orthogonally, i.e. at 90 degrees to each other), and an access port 9A built into the cylindrical sample chamber 5 for allowing access for a device for retrieving particles of interest, such as a micropipette 9. A fluid input connector 10 is shown on the base of the lens holder 7. Not visible is the fluid input orifice, of the cylindrical sample chamber 5 located in the base of the chamber. In further embodiments, the means for advancing the sample can be controlled by an external motor, such as a 4-D motor 13 (not shown in this FIG. 4) to provide movement and control in the X, Y, and Z axes, as well as to provide for rotation of the sample. It is important that the optical axes of the lenses 6A and 6B are orthogonal and co-planar such that the sample chamber and sample can be positioned at the intersection of the respective optical axes for the lenses.

In some embodiments, a cell suspension can be observed in SPIM instrument mounted in fixture and embedded in hydrogels that allow cell perfusion with fluorescently labeled antibodies, fluorescence in situ hybridization immunostaining and/or fluorescence in situ hybridization (FISH) probes, and other stains as well as media that can sustain ex vivo cell observation.

In some embodiments, the following steps are performed: Compare performance of embedding gels including agarose, collagen, polyacrylamide and tubing such as micro-perforated, fluorinated polyethylene (FPE) and glass both for fixed and live cells. Optimize fixation/permeabilization protocols. Assess need of antifading for fluorescence bleaching. Adapt SPIM image acquisition to materials chosen. Quantitative analysis of cell staining and identification or analysis of CTCs (e.g., via 3D image analysis and/or multiple antigen staining as disclosed herein).

In some embodiments, the present invention can comprise instruments and kits for the detection and characterization of CTCs and other target cell populations.

In some embodiments, a sample of cells (e.g., comprising at least one cell) of the biological sample can be analyzed by the systems and methods of the present disclosure. In some embodiments, at least one cell from a biological sample obtained from a subject can be analyzed by the systems and methods of the present invention. The biological sample can be a liquid sample, such as blood. The at least one cell can comprise at least or up to about 1 cell, at least or up to about 2 cells, at least or up to about 5 cells, at least or up to about 10 cells, at least or up to about 20 cells, at least or up to about 50 cells, at least or up to about 100 cells, at least or up to about 200 cells, at least or up to about 500 cells, at least or up to about 1000 cells, or more.

In some embodiments, the at least one cell of the biological sample can be stained with a detection moiety (e.g., a plurality of detection moieties). The detection moiety can be capable of binding to a ligand of the at least one cell. The ligand can be an extracellular ligand, a membrane-bound ligand, or an intracellular ligand. The ligand can be a small molecule, a polypeptide (e.g., a peptide or a protein), or a polynucleotide (e.g., ribonucleic acid (RNA), mRNA, deoxyribonucleic acid (DNA), etc.). The detection moiety can be an antibody. Non-limiting examples of an antibody can include a monoclonal antibody, a polyclonal antibody, a recombinant antibody, a human antibody, a humanized antibody, a Fab, a Fab′, a F(ab′) 2, an Fv, a single chain antibody (e.g., scFv), a minibody, a diabody, a single-domain antibody (“sdAb” or “nanobodies” or “camelids”), or an Fc binding domain. In some examples, the at least one cell can be treated with the detection moiety prior to being immobilized in the sample holder as disclosed herein. Alternatively or in addition to, the at least one cell can be treated with the detection moiety subsequent to being immobilized in the sample holder.

In some embodiments, the detection moiety can comprise a plurality of detection moieties that are different (e.g., multiplexing with multiple antibodies). The plurality of detection moieties can comprise at least or up to about 2 detection moieties, at least or up to about 3 detection moieties, at least or up to about 4 detection moieties, at least or up to about 5 detection moieties, at least or up to about 6 detection moieties, at least or up to about 7 detection moieties, at least or up to about 8 detection moieties, at least or up to about 9 detection moieties, at least or up to about 10 detection moieties, at least or up to about 15 detection moieties, or at least or up to about 20 detection moieties. The plurality of detection moieties can target different ligands.

In some embodiments, the plurality of detection moieties can bind a plurality of ligands that are indicative of different cell functions or cell states (e.g., different cell types, different cell origins, etc.). For examples, the plurality of ligands can be indicative different stages of cellular differentiation (or dedifferentiation). The plurality of detection moieties can comprise (i) a first detection moiety exhibiting specific binding to a first target ligand, wherein the first target ligand is a marker of a first cell type, and (ii) a second detection moiety exhibiting specific binding to a second target ligand, wherein the second target ligand is a marker for a second cell type that is different from the first cell type.

In some embodiments, different cell states (e.g., different cell types) can comprise stem cells and/or differentiated cells. Non-limiting examples different cell types (e.g., including stem cells and/or differentiated cells) can include lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells. Non-limiting examples of stem cells can include adult stem cells (e.g., mesenchymal stem cells), embdyonic stem cells, induced pluripotent stem cells, and progenitor cells (e.g., cardiac progenitor cells, neural progenitor cells, etc.).

In some embodiments, the first cell type as disclosed herein can be a differentiated cell type, such as an epithelial cell. The first ligand can comprise an epithelial cell antigen, such as epithelial cellular adhesion molecule (EpCAM) or cytokeratin (CK). In some examples, the first ligand can be one of EpCAM and CK, and the other antigen of EpCAM and CK can be bound and detected by a third detection moiety exhibiting specific binding to the other antigen. Non-limiting examples of the epithelial cell maker can include EpCam, Cadherin, Mucin-1, Cytokeratin (CK) 8, epidermal growth factor receptor (EGFR), cytokeratin (CK) 19, ErbB2, PDGF, L6, and leukocyte associated receptor (LAR).

In some embodiments, the second cell type as disclosed herein can be a stem cell type, such as a mesenchymal cell (e.g., mesenchymal stem cell). The second ligand can comprise a mesenchymal steat antigen, such as vimentin (Vim). Non-limiting examples of mesenchymal cell marker can include CD90, CD73, CD44, and vimentin.

In some embodiments, the at least one cell as disclosed herein can be detected to exhibit only one of the plurality of ligands, and such characteristic can be indicative of the at least one cell being a CTC. In some embodiments, the at least one cell as disclosed herein can be detected to exhibit two or more of the plurality of ligands, and such characteristic can be indicative of the at least one cell being a CTC. In some embodiments, a CTC from the sample of cells may be determined to have been detected when (i) a number of cells determined to exhibit two or more of the plurality of ligands is greater than or equal to (ii) a number of cells determined to exhibit only one of the two or more of the plurality of ligands. For example, a CTC associated with breast cancer may be determined to have been detected from the sample of cells when (i) a number of cells determined to exhibit two or more of the plurality of ligands (e.g., EpCAM and Vim) is greater than or equal to (ii) a number of cells determined to exhibit only one of the two or more of the plurality of ligands (e.g., EpCAM substantially alone, or Vim substantially alone).

In some embodiments, the method disclosed herein can identify different types of diseased cells. In some embodiments, the method disclosed herein can assess heterogeneity within a specific population of diseased cells. In some embodiments, the specific population of diseased cells can be CTCs, and the method disclosed herein can assess heterogeneity (e.g., different subtypes or phenotypes) within the specific population of the CTCs. In some embodiments, the method disclosed herein can assess different phenotypes or states of a population of CTCs from breast tumors. For example, the method disclosed herein can identify, distinguish, and/or quantitate (i) CTCs of mesenchymal phenotype and/or (ii) CTCs of epithelial phenotype. In another example, the method disclosed herein can identify distinguish, and/or quantitate (i) CTCs of Luminal A breast cancer, (ii) CTCs of Luminal B breast cancer, (iii) CTCs of triple-negative breast cancer, (iv) CTCs of HER2-enriched breast cancer, and/or (v) CTCs of normal-like breast cancer.

CTCs of Luminal A breast cancer can be hormone-receptor positive (e.g., estrogen-receptor and/or progesterone-receptor positive), HER2 negative, and with low levels of the protein Ki-67. CTCs of Luminal B breast cancer can be hormone-receptor positive (e.g., estrogen-receptor and/or progesterone-receptor positive), either HER2-positive or HER2-negative, and with high levels of Ki-67. CTCs of triple-negative breast cancer can be hormone-receptor negative (e.g., estrogen-receptor and progesterone-receptor negative) and HER2 negative. CTCs of HER2-enriched breast cancer can be hormone-receptor negative (e.g., estrogen-receptor and progesterone-receptor negative) and HER2 positive. CTCs of normal-like breast cancer can be hormone-receptor positive (e.g., estrogen-receptor and/or progesterone-receptor positive), HER2 negative, and with low levels of the protein Ki-67.

In some embodiments, the diseased cells as disclosed herein can be cancer cells. Non-limiting examples of cancer cells can include cells of Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, and Wilms' tumor.

In some embodiments, the CTC as detected or identified as disclosed herein may be associated with a solid tumor, such as breask cancer. In some embodiments, the CTC as detected or identified as disclosed herein may be associated with a blood cancer (e.g., non-solid tumor), such as leukemia, lymphoma, myelodysplastic syndromes (MDS), myeloproliferative disorder (MPD), and multiple myeloma.

In some embodiments, the method disclosed herein can scan a plurality of cells (e.g., millions of cells) from the blood of a subject and acquire one or more 3-dimensional cell images per cell, with resolution comparable to that of confocal microscopy, thereby enhancing the accuracy of biomarker quantitation.

The method disclosed herein can be used to identify CTCs exhibiting one or more target biomarkers. A target biomarker can be a tumor antigen (or a carcinoma-associated antigen). The tumor antigen can be encoded by a gene carrying one or more mutations. Alternatively, the tumor antigen can be encoded by a gene that does not carry a mutation. The tumor antigen can be a receptor polypeptide (e.g., a cell surface receptor polypeptide). The tumor antigen can be an ion channel, such as a cationinc ion channel for calcium singaling in a cell. In some embodiments, the tumor antigen can be a calcium signal transducer, such as Tumor-associated calcium signal transducer 2 (Trop2). In some embodiments, the tumor antigen may not be EpCAM, Vimentin (Vim), and/or Cytokeratin (CK).

CTC assessment can be a way of identifying more aggressive components of tumors. By sequencing the tumor genome in patients with metastatic breast cancer and enumerating and characterizing the CTCs present, genetic alterations that could result in higher levels of more aggressive CTCs can be identified. Additionally, if an actionable genetic alteration is found, a targeted therapy could be used in treatment with continued follow-up of CTCs over time.

Multiplex testing (e.g., 10 antibodies on a single cell) can enhance detection and detailed characterization of circulating tumor cells. The counting process can be automated.

### EXAMPLES

### Example 1: Proof-of-Concept Study for Clinical Trial Sample Analysis

The object of the present study was to determine the CTC frequency in localized and metastatic disease; to quantitate CTCs at diagnosis; timing of disappearance or persistence with treatment; to compare the CTC numbers and characteristics with information from next generation sequencing (NGS) done on the tumor biopsy in patients with metastatic disease; and to show that non-enriched CTC enumeration can compare and correlate with the epithelial capture-based, FDA-approved CellSearch® test.

### Study Population

The following patients were included in the study:

Male and female patients who were more than 18 yrs. of age with a biopsy proven diagnosis of breast cancer. All stages of patients were included in this study.

Early stage breast cancer (Stage I to III) included newly diagnosed patients before any prior treatment (surgery, chemotherapy, hormone therapy).

Patients with metastatic disease can be at initial diagnosis or at any point in treatment. Available and/or recurrent tumor samples and saliva samples from metastatic disease patients may be collected and sent to next generation sequencing testing by a commercial laboratory.

### Study Design

This study is a prospective, single arm, proof-of-concept study.

Comparison of the method disclosed herein with the FDA-approved CellSearch® technology for the detection and characterization of CTCs in peripheral blood samples from a total of 10 study patients who are either early stage or metastatic breast cancer patients.

60 (30 early stage and 30 metastatic, including the ten listed above) breast cancer patients are recruited and blood samples are collected and analyzed at study time points shown below.

### Saliva Collection Kit as Part of NGS Testing

Available and/or recurrent tumor samples from metastatic disease patients are collected and sent for next generation sequencing testing by a commercial laboratory.

### Recruitment Procedure

A total of 60 patients are enrolled in this study.

At two time points, approximately 22.5 ml peripheral blood sample are obtained, and CTCs are analyzed. The metastatic patients' saliva samples and available and/or recurrent tumor samples are collected for analysis.

### Study Procedure

At each study time point, approximately a total of 15 ml blood samples are collected from the patients, including 2×7.5 ml samples for CTC analysis.

The samples for CTC analysis are collected in Cell-Free blood collection tubes. At two study time points, approximately a total of 22.5 ml, blood samples are collected from ten patients including 2×7.5 ml samples for CTC analysis and 1×7.5 ml sample for NGS.

For the metastatic disease group, 0.65 ml of saliva sample and available and/or recurrent tumor sample are sent for NGS.

During every 3-month time points, all patients are allowed to miss 1 time point.

For the metastatic disease group, after disease progression, study patients start over with the every 3-month time point until maximum 2 years of study participation.

For the early stage disease group, study patients who have distant disease recurrence are asked to be part of the metastatic disease group.

### CTC Test Protocol:

The CTC analysis was performed on cell preparations from 15 mL aliquots of peripheral blood. Following lysis of the red blood cells, the nucleated cell pellet was collected by centrifugation, and washed and resuspended in Phosphate Buffered Saline (PBS). A cocktail of fluorescently labelled antibodies in blocking buffer was added and the cells were incubated on ice for 30 minutes. The cocktail combined CTC positive identification antibodies against EpCAM epithelial cell surface antigen, Vimentin mesenchymal cell surface antigen, CD45 leukocyte common antigen for negative selection, and combinations of therapy related markers including targeted therapy markers such as ER and HER2 depending on the patient's clinical information.

After counterstaining, the cells were thoroughly mixed with an equal volume of 2% low melting point agarose in PBS at 37° C. The cell suspension was then drawn into the appropriate specimen fixture and allowed to solidify prior to analysis. Control samples were processed in parallel to monitor the efficiency of the staining and scanning processes.

Specimens stained by immunofluorescence were analyzed using a microscopy system disclosed herein. The complete immobilized cell suspension was scanned using a 20× objective and images were digitized in 3D-image stacks. For each cell, (individual cells, cell clusters and apoptotic cells) morphological and quantitative information was obtained for each biomarker. The cells were then ranked based on morphology and quantitative information of the immunofluorescent signals.

By analyzing every nucleated cell present in a sample for quantitative immunofluorescent expression, the CTCs can be characterized as epithelial, mesenchymal, or as intermediate phenotypes.

Standard of care performance of Next Generation Sequencing: In approximately 30 patients with metastatic disease, the recurrent tumor specimen and saliva sample are sent for NGS.

### Study Time Points

Blood samples are collected at the following time points. The collection times can vary depending on treatments given. The entire study duration is 2 years for all study patients except early stage study patients who have distant disease recurrence and agree to participate in the metastatic disease group.

### Metastatic Disease Group:

At the time of enrollment to the study (+3 weeks)
   Tempus NGS saliva collection is collected at time of enrollment (+3 months)
Between 4-6 weeks after study enrollment
Every 3 months on treatment if stable disease until study completion (+/−3 weeks)
Any point at disease recurrence (+/−3 weeks window)
   Tempus NGS saliva collection may be collected at time of disease recurrence (+3 months)

Approximately 13 blood collection time points expected for this group

An additional 1×7.5 ml of blood is collected for the CellSearch® test from ten selected study patients at two of the time points as determined by the PI Early Stage Group:
At the time of enrollment to the study (+3 weeks)
Completion of initial therapy (chemotherapy/surgery) (+/−3 weeks)
Completion of secondary therapy (chemotherapy/surgery) (+/−3 weeks)
Every 3 months until study completion (+/−3-week window)
Any point of recurrence (+/−3-week window)

Approximately 13 blood collection time points are expected for this group

An additional 1×7.5 ml of blood is collected for the CellSearch® test from ten selected study patients at two of the time points as determined by the PI.

### Data Collection

Demographic information including but not limited to date of birth and gender.

Complete medical history, surgical history, social history, family history and current medications.

Imaging information related to the breast malignancy such as mammogram, ultrasound and MRI.

Complete pathology information including laterality, lymph node status, TNM staging (clinical, pathological and post neoadjuvant, DCIS or LCIS information, ER/PR status, HER2 status, Nottingham grade, Ki-67%, Miller Payne grade, lymphocytic infiltrate, lymphovascular invasion and perineural invasion) and Oncotype dx score and results of Next Generation Sequencing where appropriate.

Routine laboratory results done prior to starting treatment and after are collected.

These data include complete blood count, chemistries, tumor markers (CEA and CA15-3), germline genetic testing (i.e. BRCA), and other tests that have been performed for standard of care.

During the maximum 2 years of study participation, all standard of care and physical exam data from clinic appointments are collected.

### Study Endpoints

This Proof of Concept Study determines the potential of the liquid biopsy method disclosed herein in validating the identification of the CTCs in peripheral blood samples from breast cancer patients.

### Statistical Consideration

### Data Analysis Plan

One primary goal of this study is to validate the ability of the liquid biopsy method disclosed herein to identify CTCs in peripheral blood samples from the breast cancer patients. For each patient sample, CTC counts by different methods were obtained and compared. Pearson's correlation and Bland-Altman method were used to assess the CTC counts.

### Sample Size Justification

The sample size was calculated based on the correlation between CTC counts by the liquid biopsy method disclosed herein and CTC counts by an accredited cytogeneticist or cytopathologist. A total of 30 early-stage breast cancer patients and 30 metastatic breast cancer patients are recruited. Some of the baseline data from early stage and metastatic disease patients are used as training data set for the method disclosed herein, while the baseline data from early-stage breast cancer patients is used for validation analysis. TABLE 2 below shows predicted statistical parameters.

TABLE 2
Sample size estimation by one-sided Fisher's z test with a null
correlation of 0.6 using a one-sided α = 0.05

| # of patients | Effect size (Observed vs Null correlation) | Power |
|---|---|---|
| 30 | 0.80 vs 0.60 | 69% |
| 30 | 0.85 vs 0.60 | 91% |
| 30 | 0.90 vs 0.60 | 99% |
| 40 | 0.80 vs 0.60 | 80% |
| 55 | 0.80 vs. 0.60 | 90% |

### Example 2: Protocol for Isolation of White Blood Cells (WBCs)/CTCs from Blood Samples

### RBC Removal Utilizing Ficoll/Hypaque Protocol

1. Prepare 10% bleach solution in receptable for discarding pipettes, tubes, and tips during procedure.
2. Set out at room temperature:
   a. Hypaque 1077
   b. Hypaque 1119
   c. Sterile PBS
   d. RBC lysis buffer
   e. cell culture media (RMPI plus 10% FBS, PenStrep, glutamine)
   f. 2× freezing medium consisting of 20% DMSO in Fetal Bovine Serum (FBS)
3. Collect ˜2 mL whole human normal donor (ND) blood from each STRECK Cell-Free DNA vacutainer tube
4. Carefully transfer equal volumes (2 mL) of whole blood to each of three 15 mL conical tube labeled with sample/patient identifying number.
5. In a 15 mL conical tube, layer (e.g., all reagents at room temperature):
   a. 4 mL Hypaque 1119 (bottom layer)
   b. 4 mL Hypaque 1077 (middle layer)
   c. Mix whole blood in equal parts sterile PBS and add to 15 mL tube (top layer)
6. Centrifugation of tube at 700×g for 30 min. at room temperature
7. Two distinct layers of cells (monocytes and granulocytes respectively) form at the interfaces. Carefully remove both to a fresh 50 ml conical. The cells can then be washed and used (or subject to brief RBC lysis if necessary).
8. QS (quantity sufficient) volume of cells to 30 mL with room temperature RBC lysis buffer, mix by inverting. Label tube. Rock on speed 15 at room temperature for 10 min.
9. Add ˜25 ml of sterile, room temperature PBS to tubes. Centrifuge tubes at 300×g for 10 minutes and discard supernatant, dab tube tops on sterile towel.
10. Resuspend PBMCs for Cryopreservation in 2 ml of room temperature cell culture media (RMPI plus 10% FBS, PenStrep, glutamine).
   a. Count cells using the hemocytometer with trypan blue exclusion dye.
   b. Resuspend cells at about 2×10\hat{ }7 cells/ml in cell culture media at room temperature.
   c. Add dropwise enough 2× freezing medium at room temperature to double the volume of the cell suspension. Gently swirl the tube when adding the freezing medium.
   d. Slowly remove the cell suspension into a pipette and dispense 1 mL per cryovial
   e. Place the cryovials in a room temperature freezing container and label the container
   f. Place the freezing container as soon as possible into the −80° C. freezer
   g. Transfer the cryovials to liquid nitrogen tank after 1-14 days.
11. Fix cells by adding 500 uL of 4% paraformaldehyde in PBS, gently vortex and incubating at room temperature for 10 min., while protecting from light. Gently vortex ˜3 min. to keep cells suspended.
12. Dilute 4% PFA in QS PBS and centrifuge tubes at 300×g for 10 minutes and discard supernatant, dab tube on sterile towel.
13. Gently resuspend pellets of two tubes by pipetting with 2 mL room temperature PBS, leaving in second tube (Resuspend cells in initial whole blood volume; 2 mL of initial whole blood equates to 2 mL resuspension).
14. Dilute 20 ul of cell suspensions in 180 ul of 0.4% Trypan solution in an Eppendorf tube and count using a hemocytometer.
15. Count the 4 corner squares (16 small squares inside each) and average the value; calculate cells/ml in cell suspension. Cells/mL=average of 4 corners×10,000 (hemocytometer volume conversion factor)×10 (trypan dilution factor).

### Example 3: Antibody Staining of CTC Samples

WBC collection from previous blood draw and fixed WBC.
1. Two metastatic blood 2 mL aliquots are treated for RBC removal as stated above in EXAMPLE 2.
2. One of the samples is fixed and counted for staining protocol.
3. The other sample is cryopreserved alive for future use.

### Preparation of Reagents

1. Fixation and permeabilization Solution:
   a. Final composition is 3:1 Methanol-Glacial Acetic Acid (make fresh for each experiment)
   b. For a final volume of 4 mL, mix: 3 Methanol with 1 Glacial Acetic Acid
2. Nuclear Stain for use after hybridization:
   a. 4′,6-diamidino-2-phenylindole (DAPI) prepared in Wash Buffer A at 5 ng/mL.
   b. DAPI Stock 100 ng/ml; dilute 10 μL DAPI stock into 200 μL PBS and put ˜65 μL into each of the three tubes (when indicated below).

### Stain Cell Surface Antigens

1. Wash cells by adding 3.5 mL cold PBS/2% BSA and centrifugation at 300×g for 5 min.
2. Discard supernatant and pulse vortex to completely dissociate the pellet. (typically, 50-70 μL residual volume remains).
3. Add 2 μL FcR inhibitor and incubate tubes on ice for 5 min.
4. Add 2 μL anti-EpCAM-EBA-1-AF546, 2 μL anti-EpCAM-9C4-AF546, and 5 μL anti-CD45-AF488 to the appropriate tubes (TABLE 1) and incubate on ice for 30 min. Protect from light by covering with aluminum foil.
5. Wash with 3.5 mL PBS+2% BSA, discard supernatant by carefully decanting.
6. Permeabilize cells by adding 1 mL of methanol-acetic acid (MeOH—AcOH) fixation solution. Incubate at room temperature for 10 min.
7. Add 3.5 mL cold PBS+2% BSA. Centrifugation at 400×g for 5 min. at 4° C. Decant supernatant.
8. Gently agitate by vortex to resuspend pellet and add 250 μL of PBS/10% BSA and incubate covered for 30 min. at room temperature to block.
9. Add 2 mL PBS/2% BSA and spin by centrifugate at 400×g for 5 min. Decant supernatant (˜100 μL remains)
10. Add 2 μL anti-Vimentin-AF594 antibody, and 2 μL anti-TROP-2-AF647 antibody to appropriate tubes (TABLE 3) and incubate at room temperature for 30 min.
11. Add 3.5 mL PBS/2% BSA and spin by centrifuge at 400×g for 5 min. at 4° C. Decant supernatant.
12. Add 300 μL Hoechst 33342 nuclear stain (Diluted 1:10,000 from stock in sterile PBS) to counterstain the nuclei. Incubate in the dark at 37° C. for 30 min.
13. Add 3.5 mL cold PBS+2% BSA. Spin by centrifuge at 400×g for 5 min. at 4° C. Decant supernatant, then resuspend cells by agitating gently by vortex. Cells should be resuspended in 1-2 million WBC in ˜10 μL prior to agarose fixture.
14. Add 2 μL of 0.4 μm TetraSpeck™ microspheres (diluted from stock 1:100 in PBS) to each prior to fixture step.

TABLE 3

| | | | | EpCAM- | CD45- | | | | | |
|---|---|---|---|---|---|---|---|---|---|---|
| | | | EpCAM- | AF555 | HI30 | 90% | PBS/ | TROP2- | | |
| | | | AF555 | clone | clone- | MeOH- | 10% | F5 clone- | Vimentin- V9 | Hoechst |
| Tube | | FcR Inh. | clone 9C4 | EBA-1 | AF488 | AcOH | BSA | AF647 | clone-AF594 | 1:10,000 |
| # | Description | μL | μL | μL | μL | μL | μL | μL | μL | μL |
| 1 | All Tubes | 2 | 2 | 2 | 5 | 1000 | 250 | 2 | 2 | 300 |
| 2 | | 2 | 2 | 2 | 5 | 1000 | 250 | 2 | 2 | 300 |

### Agarose Fixture Preps

Make agarose preps using 10 μL cell solution and 10 μL 2% low-melting agarose, all combined at 37° C. and 4 μL immediately loaded in fixture.

### 1×PBS/10% BSA Solution:

For a 10% (100 mg/mL) stock solution of BSA, dissolve 4 g powdered molecular biology grade BSA in sterile PBS in a 50 ml conical flask. To avoid clumping, add 20 mL of PBS to 50 mL tube, layer BSA on surface, then add the rest of the PBS slowly dropwise. When finished, gently rock the capped tube until the BSA has dissolved completely. Pass through 0.2 μm filter.

### 2% Agarose in PBS:

Add 0.4 g low melting agarose to a 50 mL Erlenmeyer flask.

Add 20 mL of sterile PBS and swirl to make a slurry.

Heat the slurry in a microwave oven on a medium power setting until the slurry just starts to boil.

Let flask cool for several minutes, then carefully remove the flask and gently swirl to resuspend the gel particles.

Reheat the solution on a medium power setting until it just starts to boil again and let cool before use.

### 1×PBS/2% BSA Solution:

Dilute 40 mL of 1×PBS/10% BSA solution from above in 360 mL of sterile PBS.

TABLE 4
Microscope Settings

| Laser | Intensity | Exposure | Target |
|---|---|---|---|
| 405 | 20% | 10 ms | Hoechst |
| 488 | 20% | 200 ms | CD45 |
| 555 | 20% | 60 ms | EpCAM |
| 594 | 20% | 30 ms | Vimentin |
| 640 | 20% | 25 ms | TROP-2 |

Load fixture with agarose-cell immobilized suspension in Fluorescence Light Sheet Microscope.

Acquire 3-D images that can be reviewed manually for CTC detection and be utilized as for development of automated detection software with a baseline for cancer cell locations and threshold of detection.

### Example 4: Identification of TROP2 Expression in CTCs

One of the first patients enrolled in the study described in EXAMPLE 1, had metastatic triple negative breast cancer (mTNBC) and was undergoing treatment with (Trodelvy®). A first blood sample (S1) was collected, with a follow up sample (S2) collected ten weeks later. The samples were processed and stained with antibody markers suitable for assessing (i) numbers of epithelial vs. mesenchymal CTCs and (ii) whether Circulating Tumor Cells (CTC) express the cell-surface antigen Trop2.

Available Clinical Data: Time point 1 vs 2. Marked reduction in tumor markers from S1 to S2: LDH (125-220) 520 to 211; CA15-3 (0-31) 307 to 26. Marked reduction in measurable lesions on CT scan with near resolution of effusions, marked reduction in pulmonary metastasis and adenopathy; no areas of progression. Clinical resolution of palpable breast masses was not possible.

### Results

CTC frequency was high in sample S1 at ˜9.9K CTC/10⁶ WBC but dropped in S2 to ˜2.6K CTC/10⁶ WBC. CTCs were detected after immunofluorescent staining with antibodies against the cancer markers EpCAM, Vimentin (Vim) and Cytokeratin (CK). Verification of CTCs was done with positive nuclear signal detection and negative staining for CD45, only present in blood leucocytes.

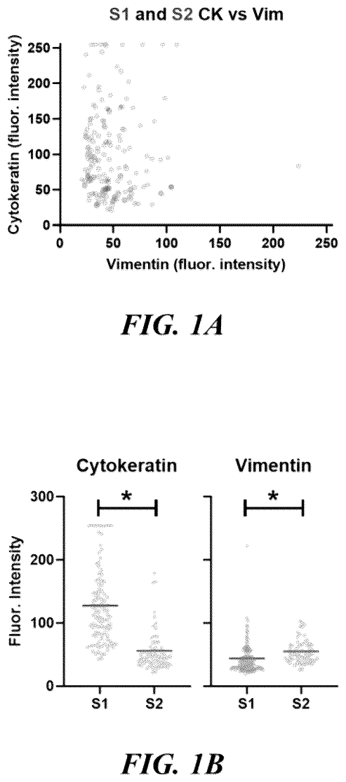

Mesenchymal vs. epithelial cell phenotype was assessed by measuring expression of VIM vs. CK in combination with expression of Trop2 (FIG. 3). Biomarker intensity measurements were done from the acquired 8-bit, 3D images where pixel grey-level values range between 0 and 255. Detected CTCs co-expressed Vim and CK. S1 CTCs expressed higher CK levels than Vim levels and the opposite was observed in S2 (FIGS. 1A and 1B).

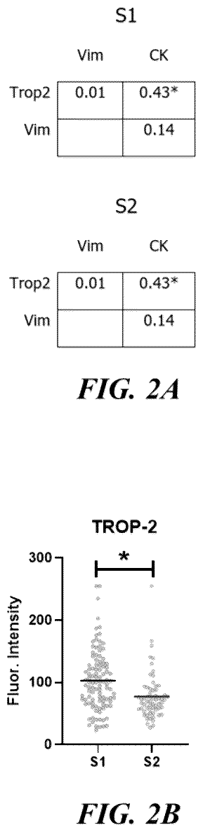

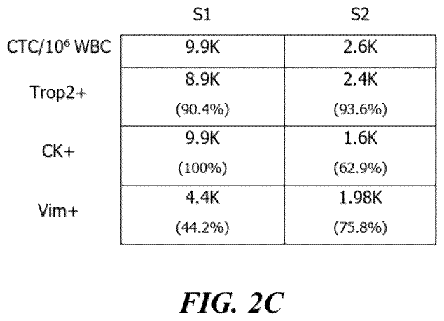

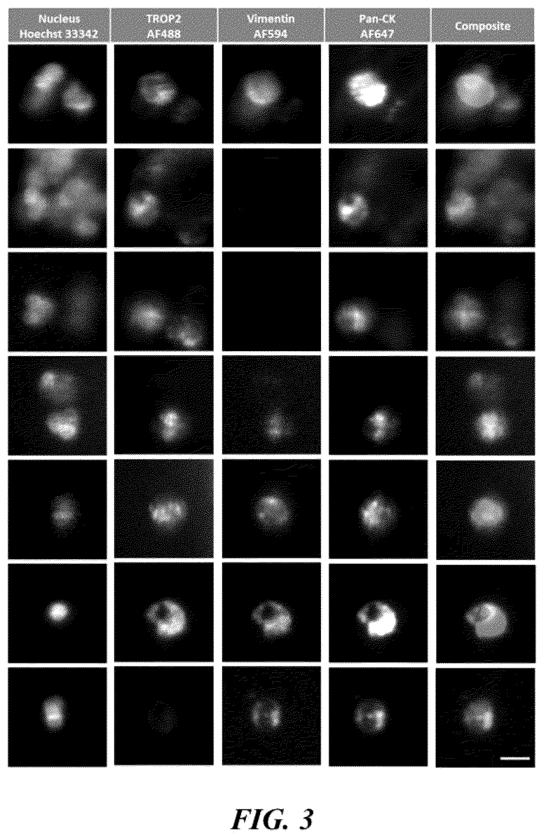

Expression of Trop2 was higher in CK+CTCs in both S1 & S2 time-points (FIG. 2A).

Comparison of Trop2 expression as a digital measurement of fluorescence intensity showed a significantly higher level in S1 (103±48.5) than in S2 (77.2±37.4), as shown in FIG. 2B. Total CTC numbers decreased between S1 and S2 by almost 75% and the total numbers of Vim+ and CK+CTCs dropped accordingly (FIG. 2C). However, the ratio of CK+/Vim+ cells changed from ˜2:1 in S1 to ˜1:1.3 in S2, at which timepoint a new subpopulation of Vim+/CK− CTCs was also observed.

The baseline sample S1 contained significant numbers of CTCs in the clinical study. S2 collected 10 weeks later, showed 75% fewer CTCs in conjunction with treatment with sacituzumab govitecan. This result correlated with significant clinical response and normalization of tumor markers and disease demonstrated on CT scans.

Trop2 co-expression in CK+ cells was observed in epithelial cells and in CTCs during tumor transition between epithelial and mesenchymal states. The expression of TROP-2 can be associated with biological aggressiveness and a poor prognosis in a number of epithelial cancers including breast, lung, and prostate. Within the detected CTC populations in each of timepoints S1 and S2, the relative percentage of Trop2+ cells remained high.

The beginning of a phenotypic reversal was also observed by comparing the relative frequencies of CK+ vs. Vim+ CTCs. In timepoint S1, a higher percentage of CK+ CTCs suggests a stronger epithelial phenotype while in timepoint S2 the higher number of Vim+ CTCs shows presence of Vim+/CK− CTCs and points to a stronger mesenchymal phenotype (FIG. 2C).

In the case of this patient, persisting Trop2 expression in S2 followed the course of the patient's disease.

### Example 5: Liquid Biopsy without Prior Enrichment

Cancer heterogeneity can utilize enrichment-free characterization of circulating tumor cells to aid in biologic understanding and clinical management. Abundant circulating breast cancer cells in 13 breast cancer patients were revealed in a liquid biopsy without prior enrichment. Liquid biopsy of 13 healthy volunteers did not reveal circulating breast cancer cells.

The presence of circulating tumor cells (CTCs) in both early and late stage breast cancer patients and the changes over time with the patient's clinical course can be demonstrated. Changes in circulating tumor cell expression of epithelial, mesenchymal, and therapeutic markers over time with a patient's changing clinical course can be demonstrated.

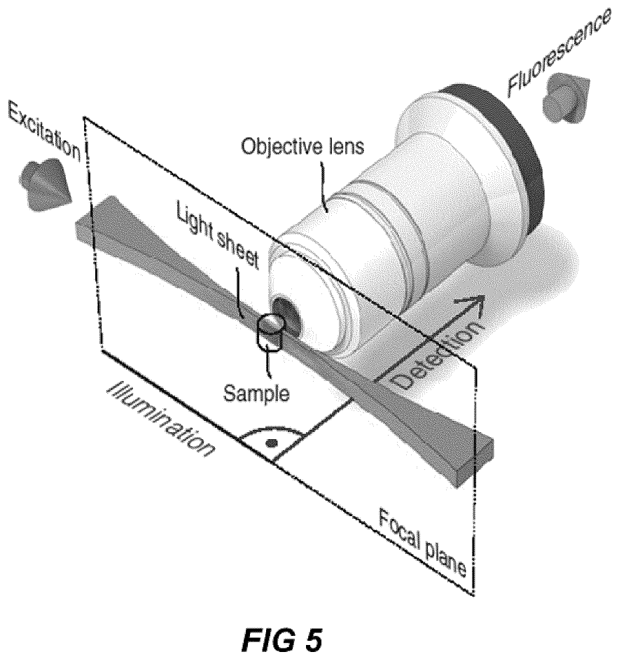

The system as disclosed herein (e.g., the RareScope system) utilized Fluorescence Light Sheet Microscopy to analyze intact, stained cells immobilized in hydrogel, with a 3D optical tomographic approach (FIG. 5).

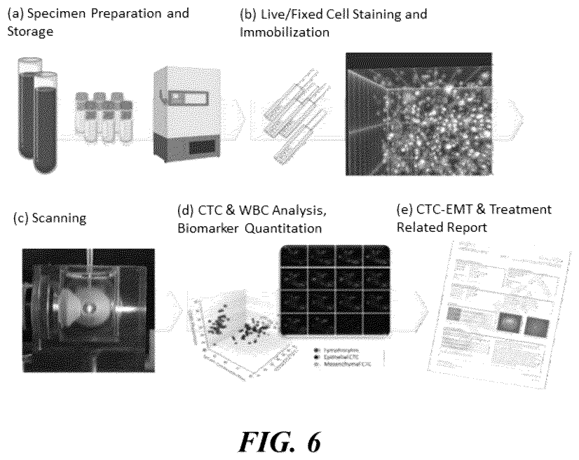

The system as disclosed herein can be a fluorescent light sheet microscopy technology for cell analysis. The system can be used to efficiently detect 2 cancer cells spiked per 1 molar nucleated cells. From each patient enrolled in the study, 18 milliliters of blood was collected and processed as shown in FIG. 6. The study can be a single site, prospective, longitudinal trial for enrichment-free, over time, CTC direction and characterization in breast cancer patients. Following red blood cell lysis, a portion of the live, nucleated cells was stored at −80° C. (FIG. 6, panel (a)) and another was fluorescently immunostained and immobilized in hydrogel within a fixture that can contain upwards of 3 million intact cells (FIG. 6, panel (b)). Cells were labeled with anti-CD45 (HI30, negative circulating tumor cell marker) and for characterization of [i] epithelial or mesenchymal phenotype anti-epithelial cell adhesion molecule (EpCAM) (9C4, EBA1), Pan-cytokeratin/CK (C11), and Vimentin/Vim (V9) monoclonal antibodies, and [ii] treatment-specific phenotype with anti-trophoblast surface antigen 2 (TROP2) (F-5), estrogen receptor-α (ER-α) (F10), and human epidermal growth factor 2 (HER2) (3B5) antibodies. Imaging utilized 6 channels to visualize fluorescently immunostained nucleated cells (FIG. 6, panel (c)). A portion of the 3D-imaged stacks was analyzed manually for expert verification of circulating tumors cells in the background of white blood cells, and thus created a ground-truth data set utilized for training machine learning automated detection software (FIG. 6, panel (d)).

About 18 milliliters of blood was collected at enrollment and at a change of treatment, or at 3-month intervals. An aliquot of all morphologically intact, un-enriched nucleated cells were placed in immobilized suspensions and analyzed. Circulating tumor cells were defined as: CD45 (−) nucleated cells stained with markers for epithelial markers, epithelial cell adhesion molecule (EpCAM), cytokeratin (CK), and/or the mesenchymal marker Vimentin (Vim). The treatment markers evaluated included trophoblast surface antigen-2 (TROP-2), estrogen receptor-α (ER-α), and human epidermal growth factor receptor 2 (HER2). Thirteen normal volunteers were tested and no circulating tumor cells were detected. In the analyzed portions of the patient's samples, a median of 43 circulating tumor cells per 1.7-2.7×10³ nucleated cells were detected (range 0-196 cells). The patient characteristics are shown in TABLE 5.

TABLE 5
Patient Characteristics

| | | Metastatic | Neoadjuvant |
|---|---|---|---|
| | | patients | patients |
| | | % (N = 10) | % (N = 3) |
| Average age at diagnosis (years) | | 53.6 | 63.7 |
| Premenopausal | | 30 | 0 |
| Postmenopausal | | 70 | 100 |
| Tumor characteristics | ER+ | 60 | 33 |
| | MER2+ | 40 | 67 |
| | TNBC | 20 | 33 |
| Stage at diagnosis | Stage I-III | 30 | 100 |
| | Stage IV | 70 | — |
| Recurrent disease | | 11.5 | — |
| (avg years since initial diagnosis) | | | |
| Treatment | Endocrine | 50 | 33 |
| | HER2-directed therapy | 40 | 63 |
| | TROP2-directed therapy | 10 | 0 |
| | Chemotherapy | 70 | 100 |

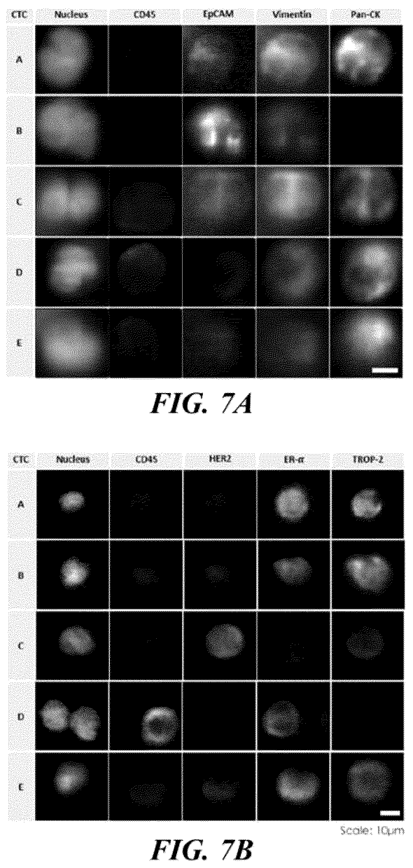

The diagnostic panel (FIG. 7A) shows expression of epithelial markers (epithelial cell adhesion molecules (EpCAM) and cytokeratin (CK)) and the mesenchymal markers (Vimentin (Vim)). Circulating tumor cells can express one or more of the three markers. Some cells expressed both mesenchymal and epithelial markers (rows A and C) and some cells only expressed one marker (rows B and E).

In the treatment panel (FIG. 7B), circulating tumor cells were analyzed for human epidermal growth factor receptor 2 (HER2), estrogen receptor-α (ER-α), and trophoblast surface antigen-2 (TROP-2) expression as presence of the markers could have determined therapeutic interventions. Circulating tumor cells can express one or more of the three markers. Cells that expressed dual markers are shown in rows A, B, and E, and cells that expressed a single marker are shown in row C and D.

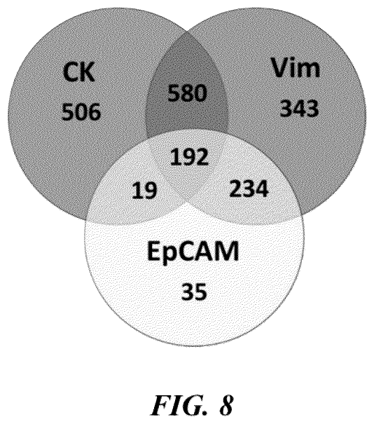

Of the 1909 total circulating tumor cells counted in all patients, 884 had only one diagnostic markers expressed (FIG. 8). Of those 884 cells, only 35 (4%) expressed epithelial cell adhesion molecule (EpCAM) as the sole marker, while 25% of the total cells were epithelial cell adhesion molecule-positive cells (EpCAM+). Out of the 884 circulating tumor cells that expressed one marker, 506 cells (57%) expressed cytokeratin (CK) as the sole marker, while 68% of the total cells expressed cytokeratin (CK). Out of the 884 circulating tumor cells that expressed one marker, 39% expressed Vimentin (Vim) as the sole marker, while 70% of total cells expressed Vimentin (Vim).

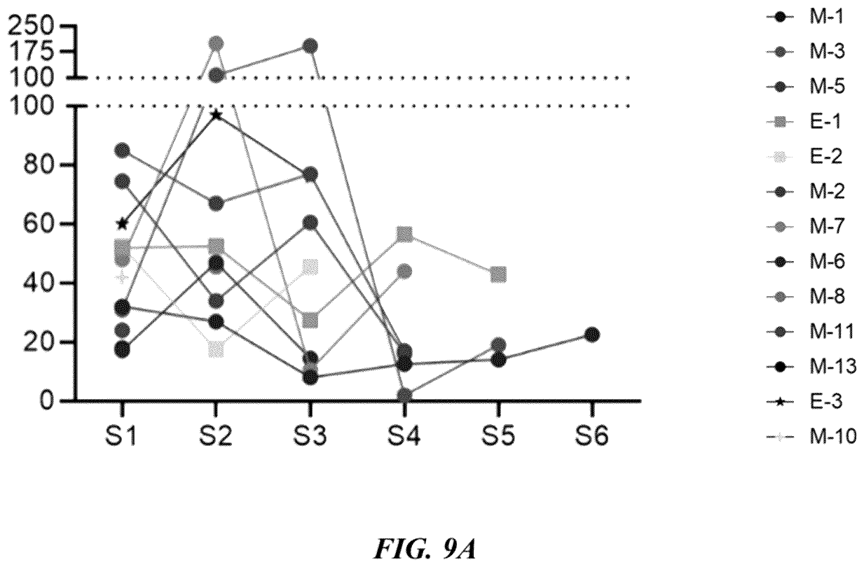

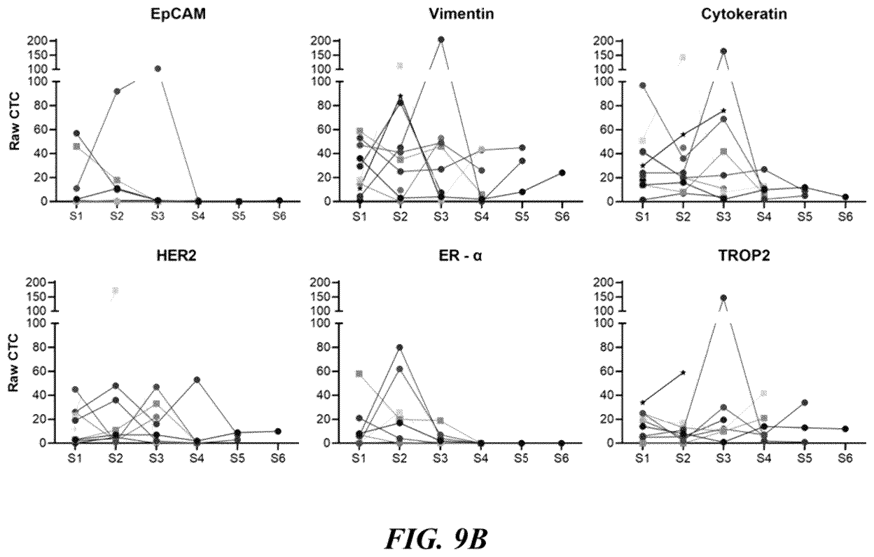

In FIG. 9A and FIG. 9A, each plot represents an individual patient and their total CTC number and phenotypic changes across time points S1 to S6.

In early and late-stage patient blood samples analyzed by the system as disclosed herein (e.g., the RareScope system), all but one sample contained circulating tumor cells at a median of 43 circulating tumor cells per 1.7-2.7×10³ nucleated cells. No circulating tumor cells were detected in samples from normal volunteers. The immunofluorescence approach was successful at identifying and characterizing circulating tumor cells by combining epithelial and mesenchymal markers.

### Example 6: Circulating Tumor Cell Counts and Phenotype in Single Breast Cancer Patient

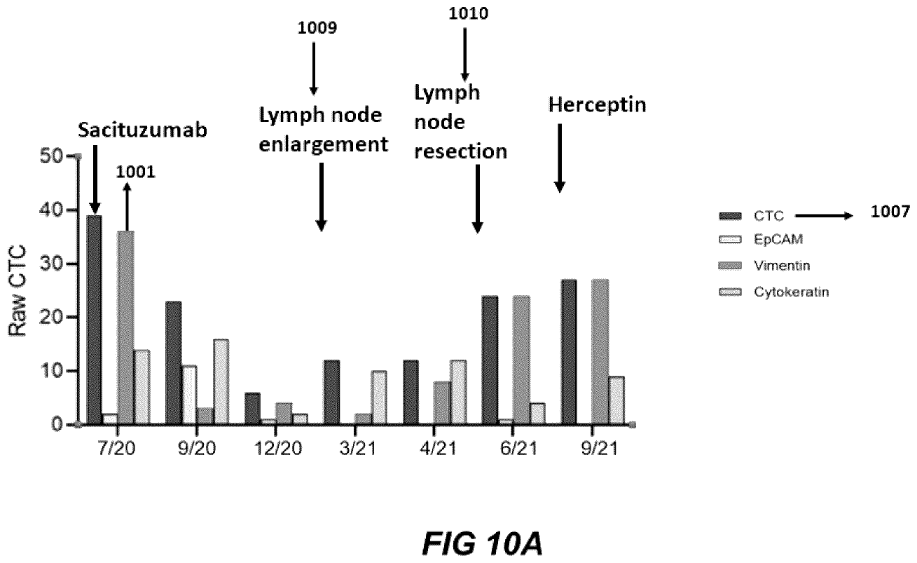

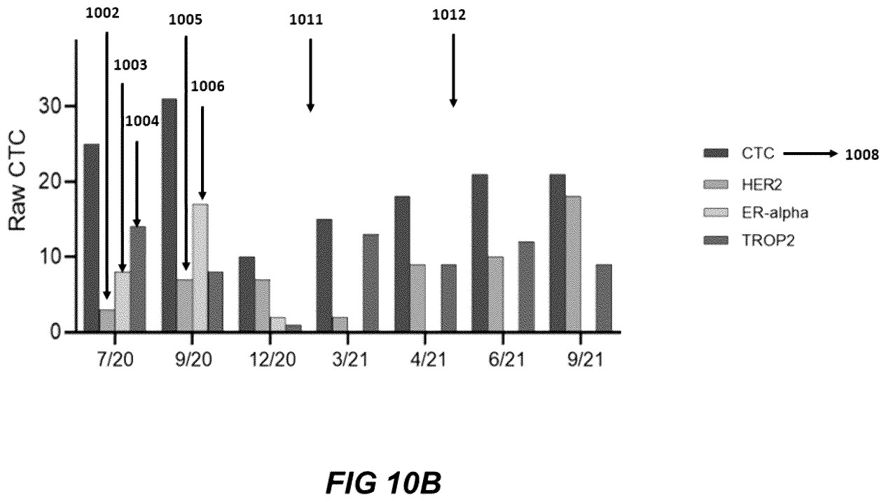

The breast cancer patient was a female patient with advanced breast disease The female patient had a breast biopsy that showed estrogen receptor-α (ER-α) poor and human epidermal growth factor receptor 2-negative (HER2−) cells. The female patient was started on chemotherapy and had a mastectomy A sample from the mastectomy showed triple negative breast cancer. Immunotherapy was added to the female patient's chemotherapy regimen. After 3 months, the female patient experienced disease progression and was enrolled in the study where a background sample was collected before the patient was started on Sacituzumab, an Antibody-Drug Conjugate therapeutic targeting TROP-2 that was approved for treatment of metastatic, triple-negative breast cancer (FIGS. 10A and 10B). Two aliquots of white blood cells were tested, one with the diagnostic panel and the other with the treatment panel. Numerous CTCs were detected (1001 in FIG. 10A) along with Her2+ (1002 in FIG. 10B) and ER-α+ (1003 in FIG. 10B) CTCs. Furthermore, TROP2+ (1004 in FIG. 10B) cells were detected. In two follow-up blood samples TROP2+ CTCs declined in numbers, and Her2+ (1005 in FIG. 10B) and ER-α+ (1006 in FIG. 10B) increased in numbers.

Local progression occurred in the axillary lymph node (1009) and resection (1010) showed human epidermal growth factor receptor 2-positive (HER2+) cells. The presence of Her2+CTCs persisted during that period of the (1011 and 1012 FIG. 10B). Trastuzumab was added to the female patient's treatment regimen.

Human epidermal growth factor receptor 2-positive (HER2+) circulating tumor cells were detected about 8 months before the human epidermal growth factor 2-positive (HER2+) lymph node biopsy that led to Herceptin initiation (about 4 months after the HER2+ lymph node biopsy) as a result of the change in cancer cell phenotype.

TROP2+CTCs were recorded before treatment with Sacituzumab was initiated and a response to treatment was observed for a 6-month period after treatment began. TROP2+ CTCs with relatively higher numbers were observed in timepoints after the diagnosis of the enlarged lymph node.

TROP2+ CTC detection can be used as a basis for a companion diagnostic to support optimization of treatment with Sacituzumab, particularly in patients where tissue biopsy is not possible.

Circulating tumor cells can be monitored over time and mirrored the patients' disease courses.

Expression of human epidermal growth factor receptor 2 (HER2), estrogen receptor-α (ER-α), and trophoblast surface antigen-2 (TROP2) varied in the same patient at different time points, which depended on patient treatment and disease progression.

The ability to delineate marker expression on circulating tumor cells in real time can be used to determine actionable change in treatment. Simultaneous expression of epithelial and mesenchymal markers can be used to demonstrate epithelial or mesenchymal transition of tumors.

### EMBODIMENTS

The following non-limiting embodiments provide illustrative examples of the invention, but do not limit the scope of the invention.

Embodiment 1. A method for analyzing a biological sample obtained from a subject, the method comprising:
(a) contacting a sample of cells of the biological sample with a plurality of detection moieties, wherein the plurality of detection moieties comprises (i) a first detection moiety exhibiting specific binding to a first target ligand, wherein the binding to the first target ligand results in staining, and (ii) a second detection moiety exhibiting specific binding to a second target ligand, wherein the binding to the second target ligand results in staining;
(b) subsequent to (a), imaging the biological sample via selective plane image microscopy, to obtain a plurality of planar images of the sample of cells, wherein the plurality of planar images comprises: (i) a first image indicative of presence or absence of the first target ligand in the sample of cells based on staining or lack of staining by the first detection moiety, and (ii) a second image indicative of presence or absence of the second target ligand in the sample of cells based on staining or lack of staining by the second detection moiety; and
(c) analyzing the plurality of planar images, to identify a diseased cell from the sample of cells,
optionally wherein:
(A) the analyzing comprises determining that a cell of the sample of cells comprises the first target ligand but not the second target ligand;
(B) the analyzing comprises determining that a cell of the sample of cells comprises the second target ligand but not the first target ligand;
(C) the analyzing comprises determining that a cell of the sample of cells comprises the first target ligand and the second target ligand;
(D) the analyzing comprises comparing (i) a distribution of the staining of the first target ligand by the first detection moiety in the first image and (ii) a distribution of the staining of the second target ligand by the second detection moiety in the second image;
(E) the diseased cell is a circulating tumor cell (CTC),
   optionally wherein:
   (1) the CTC is associated with a solid tumor, optionally wherein the CTC is associated with breast cancer; or
   (2) the CTC is associated with blood cancer; and/or
(F) the biological sample is not subjected to enrichment for the diseased cell prior to (b); and/or
(G) the first image and the second image are substantially from a common plane of the sample of cells; and/or
(H) the first image and the second image are contiguous cross-sectional images of the sample of cells; and/or
(I) the imaging comprises scanning the sample of cells with a plurality of laser sheet light sources; and/or
(J) the first detection moiety comprises an antibody or an antigen-binding fragment thereof; and/or
(K) the second detection moiety comprise an antibody or an antigen-binding fragment thereof; and/or
(L) the first cell type is a differentiated cell type,
   optionally wherein the first cell type is an epithelial cell,
      further optionally wherein:
      (1) the first target ligand comprises epithelial cellular adhesion molecule (EpCAM); and/or
      (2) the first target ligand comprises cytokeratin (CK); and/or
(M) the second cell type is a stem cell type,
   optionally wherein the first cell type is a mesenchymal cell,
      further optionally wherein the second target ligand comprises vimentin (Vim); and/or
(N) the biological sample is derived from a blood sample of the subject; and/or
(O) (1) the first target ligand is indicative of a first cell state; and (2) the second target ligand is indicative of a second cell state that is different than the first cell state, function, or type; and/or
(P) (1) the first target ligand is indicative of a first cell function; and (2) the second target ligand is indicative of a second cell function that is different than the first cell state, function, or type; and/or
(Q) (1) the first target ligand is indicative of a first cell type; and (2) the second target ligand is indicative of a second cell type that is different than the first cell state, function, or type; and/or
(R) the method further comprises repeating (a)-(c) for an additional biological sample that is obtained from the subject at a later time point than the biological sample.

Embodiment 2. A system for analyzing a biological sample obtained from a subject, the system comprising:
a container for holding a sample of cells of the biological sample;
an imaging unit configured to image the sample of cells disposed in the container via selective plane image microscopy; and
a processor operatively coupled to the imaging unit, wherein the processor is configured to:
   (a) direct the imaging unit to image the sample of cells disposed in the container, to obtain a plurality of planar images of the sample of cells, wherein the plurality of planar images comprises: (i) a first image indicative of presence or absence of a first target ligand in the sample of cells based on staining or lack of staining of the first target ligand by a first detection moiety, and (ii) a second image indicative of presence or absence of a second target ligand in the sample of cells based on staining or lack of staining of the second target ligand by a second detection moiety; and
   (b) analyze the plurality of planar images, to identify a diseased cell from the sample of cells,
optionally wherein:
(A) the processor is configured to determine that a cell of the sample of cells comprises the first target ligand but not the second target ligand; and/or
(B) the processor is configured to determine that a cell of the sample of cells comprises the second target ligand but not the first target ligand; and/or
(C) the processor is configured to determine that a cell of the sample of cells comprises the first target ligand and the second target ligand; and/or
(D) the processor is configured to compare (i) a distribution of the staining of the first target ligand by the first detection moiety in the first image and (ii) a distribution of the staining of the second target ligand by the second detection moiety in the second image; and/or
(E) the diseased cell is a circulating tumor cell (CTC),
   optionally wherein:
   (1) the CTC is associated with a solid tumor, optionally wherein the CTC is associated with breast cancer; or
   (2) the CTC is associated with blood cancer; and/or
(F) the biological sample is not subjected to enrichment for the diseased cell prior to (b); and/or
(G) the first image and the second image are substantially from a common plane of the sample of cells; and/or
(H) the first image and the second image are contiguous cross-sectional images of the sample of cells; and/or
(I) the imaging comprises scanning the sample of cells with a plurality of laser sheet light sources; and/or
(J) the first detection moiety comprise an antibody or an antigen-binding fragment thereof; and/or
(K) the second detection moiety comprise an antibody or an antigen-binding fragment thereof; and/or
(L) the first cell type is a differentiated cell type,
   optionally wherein the first cell type is an epithelial cell,
      further optionally wherein:
      (1) the first target ligand comprises epithelial cellular adhesion molecule (EpCAM); and/or
      (2) the first target ligand comprises cytokeratin (CK); and/or
(M) the second cell type is a stem cell type,
   optionally wherein the first cell type is a mesenchymal cell,
      further optionally wherein the second target ligand comprises vimentin (Vim); and/or
(N) the biological sample is derived from a blood sample of the subject; and/or
(O) (1) the first target ligand is indicative of a first cell state; and (2) the second target ligand is indicative of a second cell state that is different than the first cell state, function, or type; and/or
(P) (1) the first target ligand is indicative of a first cell function; and (2) the second target ligand is indicative of a second cell function that is different than the first cell state, function, or type; and/or
(Q) (1) the first target ligand is indicative of a first cell type; and (2) the second target ligand is indicative of a second cell type that is different than the first cell state, function, or type; and/or
(R) the processor is configured to repeat (a) and (b) for an additional biological sample that is obtained from the subject at a later time point than the biological sample.

1.-46. (canceled)

47. A method for analyzing a biological sample obtained from a subject, the method comprising:
(a) contacting a sample of cells of the biological sample with a plurality of detection moieties, wherein the plurality of detection moieties comprises (i) a first detection moiety exhibiting specific binding to a first target ligand, wherein the binding to the first target ligand results in staining, and (ii) a second detection moiety exhibiting specific binding to a second target ligand, wherein the binding to the second target ligand results in staining;
(b) subsequent to (a), imaging the biological sample via selective plane image microscopy, to obtain a plurality of planar images of the sample of cells, wherein the plurality of planar images comprises: (i) a first image indicative of presence or absence of the first target ligand in the sample of cells based on staining or lack of staining by the first detection moiety, and (ii) a second image indicative of presence or absence of the second target ligand in the sample of cells based on staining or lack of staining by the second detection moiety; and
(c) analyzing the plurality of planar images, to identify a diseased cell from the sample of cells.

48. The method of claim 47, wherein the analyzing comprises determining that a cell of the sample of cells comprises the first target ligand but not the second target ligand.

49. The method of claim 47, wherein the analyzing comprises determining that a cell of the sample of cells comprises the first target ligand and the second target ligand.

50. The method of claim 47, wherein the analyzing comprises comparing (i) a distribution of the staining of the first target ligand by the first detection moiety in the first image and (ii) a distribution of the staining of the second target ligand by the second detection moiety in the second image.

51. The method of claim 47, wherein the diseased cell is a circulating tumor cell (CTC).

52. The method of claim 51, wherein the CTC is associated with a solid tumor.

53. The method of claim 47, wherein the biological sample is not subjected to enrichment for the diseased cell prior to (b).

54. The method of claim 47, wherein the first image and the second image are substantially from a common plane of the sample of cells.

55. The method of claim 47, wherein the first image and the second image are contiguous cross-sectional images of the sample of cells.

56. The method of claim 47, wherein the imaging comprises scanning the sample of cells with a plurality of laser sheet light sources.

57. The method of claim 47, wherein the first detection moiety comprises an antibody or an antigen-binding fragment thereof.

58. The method of claim 47, wherein the second detection moiety comprise an antibody or an antigen-binding fragment thereof.

59. The method of claim 47, wherein the first cell type is a differentiated cell type.

60. The method of claim 59, wherein the first cell type is an epithelial cell.

61. The method of claim 60, wherein the first target ligand comprises epithelial cellular adhesion molecule (EpCAM).

62. The method of claim 60, wherein the first target ligand comprises cytokeratin (CK).

63. The method of claim 47, wherein the second cell type is a stem cell type.

64. The method of claim 63, wherein the first cell type is a mesenchymal cell.

65. The method of claim 64, wherein the second target ligand comprises vimentin (Vim).

66. The method of claim 47, wherein the biological sample is derived from a blood sample of the subject.
